(19) United States
(12) Patent Application Publication
(10) Pub. No.: US 2026/0199622 A1
(43) Pub. Date: Jul. 16, 2026

(54) INTEGRATED OBTURATOR DEVICE FOR EMERGENCY CRICOTHYROTOMY AIRWAY ESTABLISHMENT

(71) Applicant: 6:8 Medical Solutions LLC, Myrtle Beach, SC (US)

(72) Inventor: Uriah Shane Popp, Havre De Grace, ME (US)

(73) Assignee: 6:8 Medical Solutions LLC, Myrtle Beach, SC (US)

(21) Appl. No.: 19/447,942

(22) Filed: Jan. 13, 2026

Related U.S. Application Data
(60) Provisional application No. 63/744,556, filed on Jan. 13, 2025.

(51) Int. Cl. A61M 16/04

(52) U.S. Cl. CPC A61M 16/0497; A61M 16/044; A61M 16/0465; A61M 2210/1032

(57) ABSTRACT

A cricothyrotomy obturator for use in a cricothyrotomy procedure for establishing emergency airway access through the cricothyroid membrane. The obturator comprises a distal tip assembly having a flattened atraumatic tip configured for introduction through a surgical incision and a tapered dilating section transitioning from a flattened to a substantially circular cross-section for progressive tissue dilation. A proximal handle assembly includes syringe retention means and ergonomic finger grips. The obturator eliminates the need for separate tracheal hooks, dilators, and bougies traditionally required in cricothyrotomy procedures. The obturator is configured to receive an airway tube over its outer surface, enabling direct tracheal intubation following membrane penetration. The integrated design reduces procedural complexity, minimizes required equipment, and facilitates rapid airway establishment in emergency situations, particularly in resource-limited or field environments where conventional airway management techniques have failed.

### CROSS-REFERENCE TO RELATED APPLICATIONS

This Application claims priority to U.S. Provisional Patent Application No. 63/744,556 filed on January 13, 2025, entitled “PROCRIC,” the contents of which are incorporated herein by reference.

### BACKGROUND

### Field of the Art

The present disclosure is related to the field of medical devices, and more particularly to emergency airway management instruments designed for performing cricothyrotomy procedures.

### Discussion of the State of the Art

Surgical cricothyrotomy is an emergency airway procedure performed when conventional methods of airway management have failed or are contraindicated. This procedure involves creating an incision through the cricothyroid membrane to establish direct access to the trachea, enabling ventilation in life-threatening situations where a patient cannot be oxygenated through less invasive means. The procedure is frequently performed in trauma settings, emergency departments, pre-hospital environments, and military battlefield conditions where rapid airway establishment is essential to patient survival.

Traditional surgical cricothyrotomy techniques require the sequential use of multiple separate instruments to complete the procedure. A typical approach involves first using a scalpel to create an incision through the skin and cricothyroid membrane, followed by insertion of a tracheal hook to elevate and stabilize the membrane, then passage of a bougie or guidewire through the incision to serve as a guide for tube placement, and finally advancement of an airway tube over the bougie into the trachea. Each of these instruments must be individually retrieved, properly oriented, and manipulated within a confined anatomical space while the patient remains in critical respiratory distress.

The requirement to manage multiple separate instruments introduces substantial procedural complexity and extends the time necessary to establish a functional airway. Each transition between instruments represents an opportunity for complications, including loss of access to the incision site, inadvertent tissue trauma, and procedural delays. The tracheal hook, while intended to provide membrane stabilization, carries inherent risks of damaging surrounding anatomical structures including vascular elements and the laryngeal framework. The bougie, when advanced without adequate guidance or depth control, may penetrate the posterior tracheal wall, creating false passages or causing injury to the esophagus, or may pass through the bifurcation at the carina where it can cause a bilateral tension pneumothorax.

False passage creation represents a particularly dangerous complication wherein the airway tube is inadvertently placed outside the tracheal lumen. This may occur when the bougie or tube deviates from the intended path through the cricothyroid membrane, potentially entering subcutaneous tissues, the pretracheal space, or other structures. Once a false passage is created, subsequent attempts to establish the correct airway path become substantially more difficult due to tissue disruption and bleeding that obscures the operative field.

The procedural demands of conventional cricothyrotomy techniques require significant operator skill and fine motor control. Clinicians must maintain spatial awareness of multiple instruments while working in a small anatomical region under extreme time pressure. These psychomotor demands become particularly challenging in high-stress emergency situations where physiological stress responses may impair fine motor function. Low-visibility conditions, whether due to blood obscuring the surgical field, inadequate lighting, or environmental factors, further compound the difficulty of managing multiple separate tools.

Austere medical environments present additional challenges for conventional cricothyrotomy approaches. Military battlefield medicine, remote wilderness settings, disaster response scenarios, and resource-limited healthcare facilities may lack the full complement of specialized instruments typically available in well-equipped medical facilities. The need to assemble, organize, and sequentially deploy multiple separate tools becomes particularly problematic when equipment availability is uncertain or when environmental conditions interfere with instrument handling.

Following successful placement of the airway tube, conventional procedures require inflation of a cuff at the distal end of the tube to create a seal within the trachea. This cuff inflation step typically necessitates locating a separate syringe, connecting it to the inflation port on the tube, and delivering an appropriate volume of air to expand the cuff. During this process, the clinician must release manual control of the airway device to manipulate the syringe, creating a vulnerable interval during which the newly established airway position may be compromised. Movement of the tube during this transition, whether from patient movement, inadvertent displacement, or loss of manual stabilization, may result in dislodgement from the trachea before the airway is secured.

Existing alternative approaches to surgical cricothyrotomy have attempted to address some of these limitations with varying degrees of success. Bougie-assisted cricothyrotomy systems provide a guidewire-based approach to tube placement but continue to require the use of a separate tracheal hook to elevate and stabilize the cricothyroid membrane during the dilation and tube insertion phases. This requirement maintains the need for multiple instrument handling and preserves the associated risks of tissue damage from the hook and procedural complexity from sequential tool manipulation.

Needle-based cricothyrotomy devices offer a simplified approach that reduces the number of procedural steps, but these systems typically establish an airway of insufficient diameter to support adequate ventilation across all patient populations. The limited airway caliber achievable through needle-based techniques may prove inadequate for patients with high ventilatory demands or those requiring prolonged airway management.

Current cricothyrotomy solutions universally require the operator to manage the critical transition between airway tube placement and cuff inflation by releasing the device to locate and operate a separate syringe. This procedural gap exists across both traditional multi-instrument techniques and more recent integrated device designs, leaving a period of airway vulnerability during every cricothyrotomy procedure regardless of the specific approach employed.

### SUMMARY

The present invention provides an integrated cricothyrotomy obturator that delivers substantial clinical, operational, and practical benefits over conventional multi-instrument approaches to emergency surgical airway establishment. The obturator in accordance with the present invention includes an atraumatic distal tip, a tapered dilating section proximal to the distal tip, a flattened elongated shaft, and a syringe holder on the proximal end of the shaft.

A primary benefit of the integrated cricothyrotomy obturator in accordance with the present invention is the significant reduction in soft tissue trauma achieved through the novel atraumatic tip design. The flattened, rounded, upward-angled distal tip configuration enables passage through the surgically created cricothyroid incision without the tissue damage commonly associated with traditional tracheal hooks. By eliminating the need for a separate hook to elevate and stabilize the membrane, the invention minimizes unnecessary manipulation of delicate airway structures, thereby reducing bleeding, tissue injury, and associated complications.

The integrated cricothyrotomy obturator further provides the benefit of reduced cognitive load on medical practitioners during high-stress emergency situations. By consolidating the functions of a tracheal hook, bougie, dilator, and syringe holder into a single unified device, the integrated obturator allows practitioners to focus their attention on the life-saving procedure itself rather than on managing multiple separate instruments. This streamlined approach is particularly advantageous in emergency scenarios where split-second decisions and actions are essential to patient survival.

Another significant benefit is the enhanced procedural efficiency achieved through elimination of multiple discrete steps traditionally required in cricothyrotomy procedures. The tapered dilating section progressively enlarges the surgical opening as the device advances, obviating the need for a separate dilator instrument and its associated manipulation. The elongated flattened shaft, sized to match standard cricothyrotomy tube length and shaped to conform to the curvature of a standard cricothyrotomy tube, permits the airway tube to be advanced directly over the obturator into the trachea in a single smooth motion, substantially reducing total procedure time.

The integrated cricothyrotomy obturator further improves airway security during the critical post-insertion phase. The integrated syringe holder eliminates the common problem of a dangling syringe during the procedure and removes the requirement for the operator to release the device or reach for separate equipment to connect or inflate the tube balloon. This single-handed operation capability significantly reduces the risk of inadvertent airway loss during cuff inflation, a particularly dangerous complication that can occur when the operator must divide attention between maintaining device position and managing ancillary equipment.

The integrated cricothyrotomy obturator offers the benefit of versatile deployment across diverse clinical environments. The integrated design renders the device equally suitable for use in fully equipped hospital settings and in prehospital or austere environments where equipment availability may be severely limited. Emergency medical technicians, paramedics, flight medics, military medical personnel, and physicians in remote locations all benefit from having a single device capable of performing the complete procedure without dependence on multiple separate instruments that may be unavailable, lost, or difficult to manage under challenging field conditions.

A further benefit of the integrated cricothyrotomy obturator is the reduction in required equipment inventory and associated logistical complexity. By replacing multiple separate instruments with a single integrated device, the integrated cricothyrotomy obturator simplifies equipment procurement, storage, maintenance, and deployment. This consolidation is particularly beneficial for emergency medical services, military medical units, and remote healthcare facilities where space, weight, and equipment management constraints are significant operational considerations.

The integrated cricothyrotomy obturator additionally provides the benefit of flexible implementation options to meet varying clinical and institutional requirements. The device may be manufactured from medical-grade polymers or metals and may be configured as either single-use disposable or reusable depending on implementation requirements and sterilization capabilities. This adaptability allows healthcare providers to select the configuration most appropriate for their specific operational context and infection control protocols.

The integrated cricothyrotomy obturator further provides the benefit of comprehensive kit availability, wherein the integrated obturator may be provided as part of a complete cricothyrotomy kit including the obturator, a 6.0 mm cricothyrotomy or other sized tube, neck strap, syringe, and scalpel. This kit configuration ensures that all necessary components for emergency surgical airway establishment are immediately available in a single package, eliminating the need to gather separate items during a critical emergency and further reducing procedure initiation time.

In one example, the present invention is an obturator for a cricothyrotomy procedure. The obturator includes an elongated shaft comprising a proximal portion having a proximal end and a distal portion having a distal end. The shaft is curved, and the distal portion has a cross-sectional shape that is flat. The proximal portion of the shaft may have a second cross-sectional shape that is different from the distal portion cross-sectional shape. For example, the second cross-sectional shape may be a cross. The cross-sectional shape of the distal portion may be a solid, flat rectangle.

The obturator further includes a distal tip assembly comprising a tapered dilating section directly coupled to the distal end of the shaft, and an atraumatic tip directly coupled to the tapered dilating section. The atraumatic tip may have a solid, flat cross-section and a rounded end. The tapered dilating section may have a cross-sectional shape that gradually increases from a first size that is substantially equal to that of the flat cross-section to a second size having a round cross-section. The distal tip assembly may be formed as a single, unitary piece. The distal tip assembly and the elongated shaft may be formed as a single, unitary piece.

The obturator further includes a proximal handle assembly comprising a finger hold handle directly coupled to the proximal end of the shaft, and a syringe holder directly coupled to the finger hold handle. The syringe holder has a cylindrical bore configured for retaining a syringe barrel therein. The proximal handle assembly may be formed as a single, unitary piece.

In another example, the present invention is an assembly for a cricothyrotomy procedure. The assembly includes a cricothyrotomy tube, an obturator configured for being positioned within the cricothyrotomy tube, and a syringe operatively coupled to the cricothyrotomy tube and configured for inflating a cuff on the cricothyrotomy tube. The obturator includes an elongated shaft that is sized and shaped to fit entirely within the cricothyrotomy tube. The cricothyrotomy tube may be curved and the elongated shaft may have a curved profile that matches that of the cricothyrotomy tube. The elongated shaft of the obturator may have a proximal section comprising a first cross-sectional shape and a distal section comprising a second cross-sectional shape that is different from the first cross-sectional shape. The first cross-sectional shape may be a cross and the second cross-sectional shape may be solid and flat

The obturator further includes a distal tip assembly comprising a tapered dilating section directly coupled to a distal end of the shaft, and an atraumatic tip directly coupled to the tapered dilating section. The atraumatic tip has a solid, flat cross-section and a rounded end. The tapered dilating section has a cross-sectional shape that gradually increases from a first size that is substantially equal to that of the flat cross-section to a second size that is round and has a diameter approximately equal to an inner diameter of the cricothyrotomy tube. The distal tip assembly may be formed as a single, unitary piece. The distal tip assembly and the elongated shaft may be formed as a single, unitary piece.

The obturator further includes a proximal handle assembly comprising a finger hold handle directly coupled to a proximal end of the shaft, and a syringe holder directly coupled to the finger hold handle. The syringe holder has a cylindrical bore having an inner diameter that is approximately equal to an outer diameter of a barrel of the syringe. The proximal handle assembly may be formed as a single, unitary piece.

The assembly may have an assembled configuration wherein the elongated shaft of the obturator may be positioned within the cricothyrotomy tube, the syringe may be positioned in the syringe holder, the distal tip assembly of the obturator may protrude out of a distal end of the cricothyrotomy tube, and the proximal handle assembly may be positioned proximally of a proximal end of the cricothyrotomy tube.

These and other benefits, features, and advantages of the present invention will become more apparent from the following detailed description when taken in conjunction with the accompanying drawings.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 is a front elevation view of an obturator for use in a cricothyrotomy procedure, in accordance with an embodiment of the present invention.

FIG. 2 is a perspective view of an obturator for use in a cricothyrotomy procedure, in accordance with an embodiment of the present invention.

FIGS. 3A and 3B are cross-section views of a shaft of an obturator taken along lines A-A and B-B of FIG. 2, respectively, in accordance with an embodiment of the present invention.

FIG. 4 is a close up view of a distal end of an obturator protruding from a distal end of a cricothyrotomy tube, in accordance with an embodiment of the present invention.

FIG. 5 illustrates the use of a finger hold handle that is attached to a proximal end of an obturator, in accordance with an embodiment of the present invention.

FIGS. 6 and 7 are perspective views of a cricothyrotomy assembly in a disassembled configuration and an assembled configuration, respectively, in accordance with an embodiment of the present invention.

### DETAILED DESCRIPTION

The present invention is an obturator for a cricothyrotomy procedure. The obturator is configured for being used with a cricothyrotomy tube having a syringe operatively coupled thereto. The obturator includes an elongated flattened shaft, a distal tip assembly coupled to the distal end of the shaft and a handle assembly coupled to the proximal end of the shaft. The distal tip assembly includes an atraumatic distal tip and a tapered dilating section positioned proximally to the distal tip. The handle assembly includes a finger hold handle and a syringe holder configured for retaining a syringe in a fixed position during the cricothyrotomy procedure.

The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non- simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Apparatus

The integrated cricothyrotomy obturator of the present invention comprises a unitary elongated instrument configured for facilitating emergency surgical airway access through the cricothyroid membrane. The device integrates multiple functional components that were previously provided as separate instruments, thereby reducing the equipment burden and procedural complexity associated with emergency cricothyrotomy procedures.

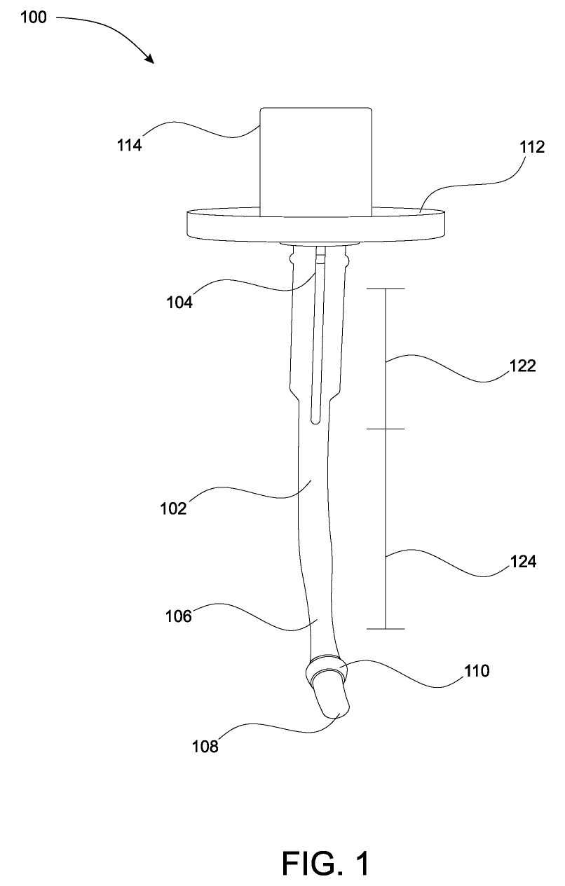

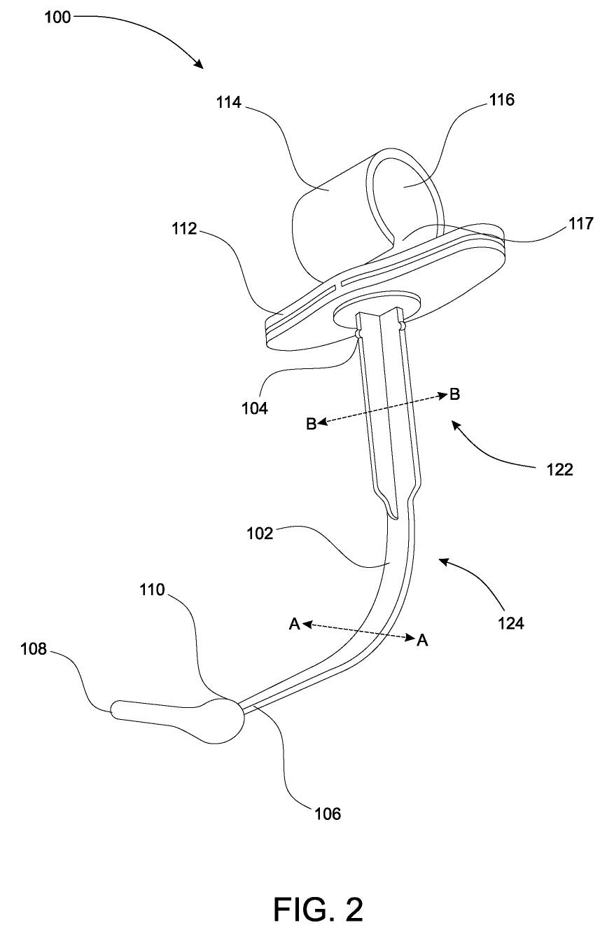

In general, as shown in FIGS. 1 and 2, the cricothyrotomy obturator 100 includes an elongated flattened shaft 102 having a proximal end 104 and a distal end 106. The distal end 106 of the shaft 102 has a distal tip assembly that includes an atraumatic distal tip 108 and a tapered dilating section 110 extending proximally from the distal tip 108. The proximal end 104 of the shaft 102 is coupled to a proximal handle assembly that includes a finger hold handle 112 fixedly coupled to a syringe holder 114.

The atraumatic distal tip 108 has a solid, flattened cross-sectional profile and a rounded tip. The solid, flattened cross-sectional profile departs from conventional cylindrical configurations found in standard airway management devices. This flattened geometry of the distal tip 108 has a reduced vertical height relative to its lateral width, thereby enabling passage through minimal incision dimensions while maintaining structural integrity sufficient for tissue manipulation. The rounded tip of the atraumatic distal tip 108 distributes contact forces across a broader tissue interface area, thereby reducing localized pressure concentrations that might otherwise cause tissue trauma, perforation, or laceration during insertion. The distal tip 108 is solid and is therefore free from openings, lumens, and protrusions.

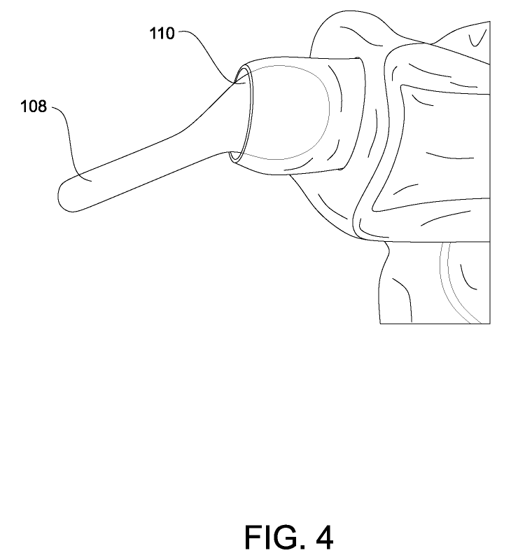

The distal tip 108, shown in more detail in FIG. 4, further has an upward angular orientation configured to facilitate initial insertion through a surgically created cricothyroid membrane incision without causing tissue trauma or requiring a separate tracheal hook for membrane elevation. During use, the patient is typically in a supine position, and the distal tip 108 is angled upwards relative to the patient. The angular orientation enables the distal tip 108 to naturally lift and separate the edges of the cricothyroid membrane incision during insertion, thereby eliminating the requirement for a separate tracheal hook instrument. The lifting action provided by the angular orientation allows the clinician to insert the device 100 through a horizontal stab incision while simultaneously creating vertical displacement of the membrane edges, facilitating passage of the wider portions of the device 100. The upward angular orientation directs advancement forces in a vector that separates the anterior membrane tissue from underlying structures, thereby creating a working space for continued device insertion without requiring manual tissue manipulation by the operator.

The peripheral edges of the atraumatic distal tip 108 have rounded contours that prevent cutting, scoring, or tearing of membrane tissue during insertion and manipulation. The absence of sharp edges or angular transitions reduces the likelihood of tissue damage during both insertion and any repositioning movements that may occur during the procedure.

The atraumatic distal tip 108 may be manufactured from medical-grade polymeric materials including, but not limited to, polyethylene, polypropylene, or polyether ether ketone (PEEK). In embodiments intended for reusable applications, the atraumatic distal tip 108 may be fabricated from polished stainless steel alloys or titanium alloys that can withstand repeated sterilization cycles. The atraumatic distal tip 108 may have a smooth surface finish that minimizes frictional resistance during advancement through tissue and prevents tissue fibers from catching or snagging on surface irregularities. In certain embodiments, the atraumatic distal tip 108 may incorporate surface treatments such as hydrophilic coatings that reduce insertion friction by promoting a lubricating fluid layer at the tissue-device interface.

The atraumatic distal tip 108 may be integrally formed with adjacent device components, particularly the tapered dilating section 110, to create a continuous surface topology without joints, seams, or discontinuities where tissue could become entrapped or damaged. In other words, the atraumatic distal tip 108 and the tapered dilating section 110 may be a single, unitary piece. This integral construction eliminates potential failure points and ensures consistent performance characteristics across the transition region between the atraumatic distal tip 108 and adjacent structures.

The tapered dilating section 110 extends proximally from the distal tip 108 and terminates at the junction with the main shaft 102 of the obturator 100, defining a continuous geometric transition zone. The tapered dilating section 110 is configured to progressively enlarge the cricothyroid membrane incision from the initial entry dimension to the full diameter required for cricothyrotomy tube passage. The cross-sectional shape of the tapered dilating section 110 gradually increases from the flattened tip geometry of the distal tip assembly 108 to a round cross-section that is slightly smaller than, or approximately equal to, the inner diameter of the cricothyrotomy tube. In one embodiment, the tapered dilating section 110 comprises a proximal terminus having a circular cross-section with a diameter of approximately 6 to 10 millimeters, corresponding to the inner diameter of standard cricothyrotomy tubes. This dimensional relationship ensures that tissue dilation achieved by advancement of the tapered dilating section 110 produces an opening of adequate size to receive the cricothyrotomy tube without requiring additional dilation. The taper angle of the dilating section 110 is configured to provide gradual dilation of the membrane incision, which distributes the dilating forces over a greater tissue contact area, reducing the peak stress applied to the membrane edges and minimizing the risk of tissue tearing or extension of the incision beyond the intended boundaries.

The distal tip assembly, including the tapered dilating section 110 and the adjacent distal tip 108 may be formed as a unitary, monolithic structure with the elongated shaft 102. This integral construction eliminates discontinuities, joints, seams, or interfaces along the outer surface that might otherwise engage or catch tissue during advancement. Making the tapered dilating section 110 of the same material as adjacent obturator components facilitates manufacturing efficiency and provides consistent mechanical properties throughout the assembly. Suitable materials include medical-grade polymeric materials, stainless steel alloys, or other biocompatible materials exhibiting appropriate rigidity and surface characteristics. The tapered dilating section 110 may have a smooth surface finish configured to minimize frictional resistance during advancement of the device 100 through tissue. In certain embodiments, the tapered dilating section 110 may include a lubricious coating applied to the outer surface to further reduce advancement resistance.

During operation, as a clinician advances the obturator 100 through an initial membrane incision, the tapered dilating section 110 engages the tissue margins and progressively expands the opening. The gradual dimensional increase of the tapered dilating section 110 permits controlled advancement with reduced force requirements compared to abrupt diameter transitions.

The tapered dilating section 110 is not limited to the illustrated shape. Rather, the tapered dilating section 110 may incorporate variations in the taper geometry, including linear tapers, parabolic tapers, or compound curves optimized for specific tissue characteristics or tube dimensions. The cross-sectional transition may alternatively progress through other intermediate geometries, such as stadium-shaped or rectangular profiles with rounded corners, depending on the configuration of the distal tip 108.

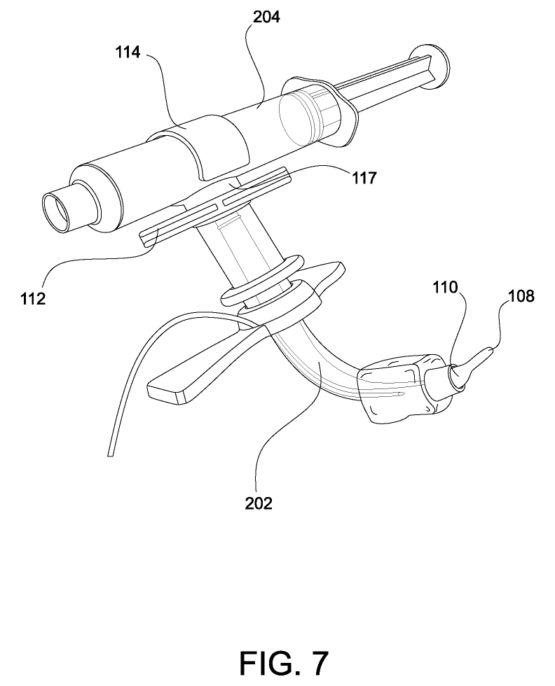

The elongated flattened shaft 102 provides the structural backbone of the obturator 100 and serves as a guide rail over which the cricothyrotomy tube advances into the trachea. The elongated flattened shaft 102 extends between the tapered dilating section 110 and the proximal handle 112. The shaft 102 has a length that is approximately equal to the standard length of a cricothyrotomy tube, and has a curved profile conforming to the curved profile of the cricothyrotomy tube. As shown in FIG. 7, substantially the entire length of the shaft 102 is disposed within the cricothyrotomy tube 202 when the device 100 is in use.

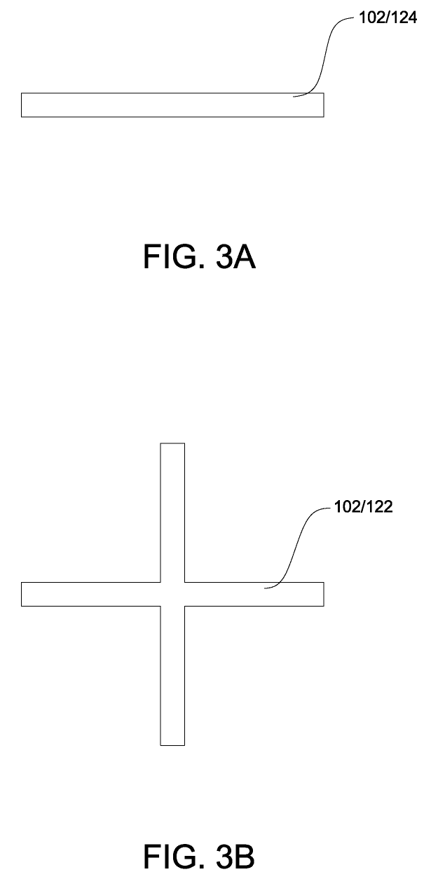

The elongated flattened shaft 102 has a straight proximal portion 122 and a curved distal portion 124. The proximal portion 122 and the distal portion 124 may have different cross-sectional shapes. At least a portion of the shaft 102 has a cross-sectional geometry characterized by a major dimension exceeding a minor dimension, thereby establishing the flattened configuration. The cross-sectional configuration of the elongated flattened shaft 102 comprises a flattened rectangular, elliptical, or oval shape. This flattened configuration provides structural rigidity against bending in the direction of the minor axis while permitting controlled flexibility in the direction of the major axis. The flattened shaft configuration further facilitates visual and tactile confirmation of device orientation during insertion, as the clinician can readily determine the rotational position of the device by observing or palpating the cross-section of the shaft 102. An example of the cross-sectional shape of the flattened shaft is shown in FIG. 3A, which depicts the cross-sectional shape as being a flat, solid rectangle. The solid shaft 102 is free from lumens or openings.

The curved profile of the elongated flattened shaft 102 conforms to the anatomical curvature of the airway from the cricothyroid membrane insertion point to the tracheal lumen. In one embodiment, the concave surface of the shaft 102 is oriented anteriorly when the device 100 is properly positioned. This curvature matches the bend incorporated into standard cricothyrotomy tubes and ensures that the tube follows the intended path when advanced over the obturator shaft 102. The elongated flattened shaft 102 has a curved profile along at least a portion of its longitudinal extent. In certain embodiments, the curved profile comprises an angular deviation in the range of approximately 30 to approximately 45 degrees relative to a proximal longitudinal axis. The flattened portion of the shaft 102 may be configured to bend and then return to the manufactured shape. The flexibility of the shaft 102 facilitates removal of the obturator 100 from the cricothyrotomy tube.

The connection between the elongated flattened shaft 102 and the tapered dilating section 110 comprises a smooth transition zone wherein the shaft cross-section gradually increases in dimension to match the proximal terminus of the tapered dilating section 110. The shaft 102, dilating section 110 and atraumatic tip 108 may be manufactured as a single, unitary piece. Alternatively, the shaft 102, dilating section 110 and distal tip 108 may be manufactured separately and then fixedly attached together.

The flattened cross-sectional geometry of the elongated flattened shaft 102 provides enhanced resistance to column buckling relative to a circular cross-section of equivalent cross-sectional area. In certain embodiments, the cross-sectional geometry of the elongated flattened shaft 102 transitions along its longitudinal extent. The distal portion 124 of the elongated flattened shaft 102 may exhibit the flattened cross-sectional configuration, while the proximal portion 122 transitions to a reinforced section at the junction with the handle 112. This transitional geometry accommodates interface requirements with adjacent components while maintaining the flattened configuration along the functional guiding length. The reinforced section may have a cross sectional shape that is cross-shaped, as shown in FIG. 3B. However, other cross-sectional shapes of the reinforced section are within the scope of the present invention. For example, the reinforced section 122 may have a cross-sectional shape that is square, rectangular, circular, oval, elliptical, triangular, or the like.

The elongated flattened shaft 102 may be manufactured from medical-grade materials selected to provide a combination of structural rigidity and controlled flexibility. Suitable materials include reinforced polymer compositions, austenitic stainless steel alloys, and nickel-titanium shape memory alloys. The shaft 102 may be made of the same material as the dilating section 110 and the atraumatic tip 108. The material selection and cross-sectional dimensions are configured to provide sufficient rigidity to resist buckling under axial insertion forces while permitting the elongated flattened shaft 102 to conform to the curved anatomical pathway without permanent deformation.

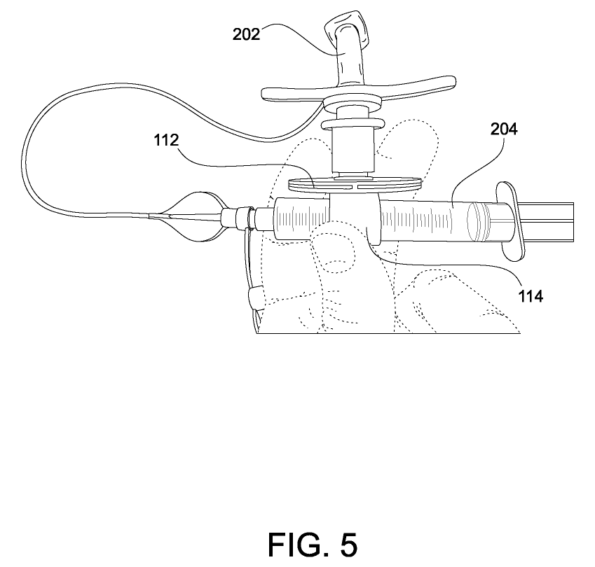

The proximal finger hold handle 112 has an ergonomically designed gripping interface located at the proximal end 104 of the shaft 102, and is configured to provide secure manual control and single-handed operation during device insertion, advancement, and withdrawal. The finger hold handle 112 incorporates one or more finger engagement features configured to provide secure grip and prevent slippage during manipulation. In one embodiment, the finger engagement features comprise a pair of lateral finger rests positioned on opposite sides of the body of the handle 112. Each finger rest may have a concave surface contoured to receive the pad of a finger. The finger rests are positioned to accommodate the index finger and middle finger of an adult hand, as depicted in FIG. 5. The proximal finger hold handle 112 provides a mechanical interface between the operator’s hand and the elongate body of the device 100, enabling transmission of axial forces, rotational inputs, and tactile feedback throughout the insertion sequence.

The proximal finger hold handle 112 may alternatively or additionally comprise contoured grip surfaces featuring raised flanges, recessed channels, or sculpted regions that conform to the natural grasp pattern of the human hand. These contoured surfaces are arranged to receive the palmar and digital surfaces of the operator’s hand in a manner that promotes stable retention even when the operator is wearing medical examination gloves, surgical gloves, or other protective hand coverings that may reduce tactile sensitivity and friction coefficients. Alternatively or additionally, the finger hold handle 112 may comprise circumferential ridges, textured surfaces, or elastomeric overmolding to enhance grip security.

The external surfaces of the proximal finger hold handle 112 may incorporate textured regions comprising knurled patterns, cross-hatched configurations, stippled surfaces, ridged formations, or elastomeric overmolded zones that enhance the coefficient of friction between the handle 112 and the operator’s gloved or ungloved hand. The textured regions may be disposed across substantially the entire gripping surface of the handle 112 or may be selectively positioned at high-contact zones where slip prevention is particularly advantageous.

The geometric configuration of the proximal finger hold handle 112 is arranged to distribute insertion forces across multiple digits of the operator’s hand, thereby reducing fatigue accumulation in any single digit and enabling sustained precise control over device advancement speed, insertion depth, and rotational orientation. The force distribution characteristics of the handle 112 permit the operator to apply controlled axial pressure while simultaneously maintaining fine motor control for navigating anatomical structures.

The angular orientation of the proximal finger hold handle 112 relative to the longitudinal axis of the device is selected to align the operator’s hand position with a natural insertion trajectory, wherein this alignment reduces biomechanical strain on the operator’s wrist, forearm, and hand during the insertion procedure. The handle orientation may be configured such that the operator’s wrist remains in a substantially neutral position throughout the manipulation sequence, thereby promoting improved fine motor control and reducing operator fatigue.

The proximal finger hold handle 112 is configured to function as a mechanical stop that limits distal travel of the obturator 100 relative to the outer cannula or sheath, thereby preventing over-insertion of the obturator 100 beyond a predetermined depth. The stop function may be accomplished through abutment of the handle 112 against a corresponding surface of the outer cannula assembly or through engagement with a complementary stop feature disposed on an adjacent component.

The proximal finger hold handle 112 may be manufactured from medical-grade polymeric materials including but not limited to polycarbonate, acrylonitrile butadiene styrene, polyetheretherketone, polypropylene, polyethylene, or copolymer formulations thereof. The polymeric materials may be selected to exhibit mechanical properties suitable for repeated sterilization cycles while maintaining dimensional stability and structural integrity. The materials comprising the proximal finger hold handle 112 may be selected for compatibility with sterilization modalities.

In certain embodiments, the proximal finger hold handle 112 comprises a base substrate formed from a rigid material with a textured overmolding layer formed from a softer elastomeric material such as thermoplastic elastomer, silicone, or santoprene disposed over at least a portion of the gripping surfaces. The overmolding layer enhances grip characteristics while the rigid substrate provides structural support for force transmission.

The finger hold handle 112 is not limited to the illustrated shape. Alternative configurations for the proximal grip region may include ring handle arrangements similar to those employed in surgical scissors or hemostats, wherein one or more annular apertures receive the operator’s digits. Pistol-grip handle configurations wherein the handle extends at an acute or obtuse angle relative to the device axis represent a further alternative approach. Pistol-grip arrangements may provide ergonomic advantages for certain manipulation tasks but may add dimensional bulk that interferes with operation within the confined anatomical space at the patient’s neck region during airway management procedures.

The integrated syringe holder 114 is configured to receive and securely retain a standard medical syringe in a readily accessible position for immediate cuff inflation without requiring the clinician to release the device 100 or reach for separate equipment. The integrated syringe holder 114 comprises a receptacle formed in or attached to the proximal finger hold handle 112. The receptacle is dimensioned to receive the barrel of a standard Luer-lock or Luer-slip syringe, and is configured to releasably secure a syringe in a predetermined orientation relative to the obturator shaft 102 and finger hold handle 112.

The syringe holder receptacle may comprise a cylindrical bore 116 (shown in FIG. 2) having an inner diameter of approximately 10 to 15 millimeters and a width of approximately 15 to 25 millimeters, sized to receive syringe barrels having a capacity of 5 to 20 milliliters. The syringe barrel is retained in the cylindrical bore 116 by friction fit, but other retention features configured to hold the syringe are within the scope of this invention. For example, syringe retention mechanisms may include one or more of clips, friction-fit channels, snap-lock features, detent structures, or elastomeric gripping elements that engage the syringe barrel with sufficient force to resist dislodgement during normal handling while permitting rapid removal when necessary. The retention force is calibrated to balance security against ease of extraction, enabling the clinician to withdraw the syringe without requiring excessive force or complex manipulation. The integrated syringe holder 114 may incorporate universal sizing features including adjustable width channels, compliant gripping surfaces, or multiple engagement positions to accept syringes manufactured by different suppliers, thereby reducing compatibility constraints during emergency use scenarios where specific syringe brands may not be available.

As shown in FIGS. 2 and 7, the cylindrical bore 116 may not be completely enclosed. That is, the syringe holder 114 may include an opening 117, such that the syringe holder 114 has one side that is fixedly coupled to the finger hold handle 112 and an opposite side that is not attached and spaced apart from the finger hold handle 112 to create the opening 117. The syringe holder 114 may have a cross-section that is substantially C-shaped. The syringe holder 114 may be made of a semi-flexible material so that the syringe 204 can be removed through the opening 117 and the syringe holder 114 can return to it’s manufactured shape (shown in FIG. 2) after the syringe is removed. In this manner, the syringe holder 114 can flex to accommodate syringe barrels of different sizes

The syringe holder 114 is positioned to place the syringe within reach of the clinician’s thumb or fingers while the hand remains in the grip position on the finger hold handle 112. This positioning enables the clinician to inflate the cuff without releasing grip on the obturator 100 or requiring assistance from a second clinician. The integrated syringe holder 114 maintains the syringe in a position that facilitates single-handed access during airway management procedures while preventing inadvertent displacement during device manipulation and insertion.

The integrated syringe holder 114 positions the retained syringe in an orientation substantially perpendicular to or at an acute angle relative to the longitudinal axis of the obturator shaft 102. This spatial relationship places the syringe plunger within the sweep arc of the thumb of a hand gripping the finger hold handle 112, thereby enabling the clinician to access and operate the syringe without releasing grip on the device 100 or reaching away from the procedural field. The angular orientation of the integrated syringe holder 114 may be optimized through ergonomic analysis to accommodate variations in hand size and grip style while maintaining the syringe clear of the insertion trajectory.

The integrated syringe holder 114 may be formed integrally with the handle 112 using common materials and manufacturing processes, such as injection molding of medical-grade polymers. Alternatively, the integrated syringe holder 114 may comprise a separate component attached to the handle 112 through mechanical fastening, adhesive bonding, ultrasonic welding, or snap-fit engagement. The material selection for the integrated syringe holder 114 provides sufficient structural rigidity to maintain syringe position while incorporating compliance in the retention features to permit syringe insertion and removal.

The integrated syringe holder 114 is configured such that the retained syringe does not protrude into regions that would interfere with device insertion through the cricothyroid membrane or subsequent advancement of the airway tube. The spatial envelope occupied by the syringe when secured in the integrated syringe holder 114 is offset from the working axis of the device 100 to maintain clearance during procedural manipulation.

In kit configurations, the integrated syringe holder 114 may be pre-loaded with a syringe 204 as shown in FIGS. 5-7. The syringe 204 may be pre-filled with a measured volume of air corresponding to the recommended cuff inflation volume. The integrated syringe holder 114 permits multiple insertion and removal cycles, accommodating situations where cuff pressure adjustment or integrity verification requires repeated syringe access.

The integrated cricothyrotomy obturator 100 may be manufactured from medical-grade materials suitable for contact with internal tissues and bodily fluids. Suitable materials include stainless steel alloys, titanium alloys, and medical-grade polymers such as polycarbonate, polyetheretherketone, acrylonitrile butadiene styrene, or the like, or a combination thereof. The device 100 may be manufactured as a single integral component through machining, molding, or additive manufacturing processes, or may be assembled from multiple components joined through welding, adhesive bonding, or mechanical fastening.

In one embodiment, the atraumatic distal tip assembly 108, tapered dilating section 110, and elongated flattened shaft 102 are formed as an integral unit from a single material, with the proximal finger hold handle 112 attached as a separate component. The handle 112 may be overmolded onto the proximal end 104 of the shaft 102, providing a secure mechanical connection while permitting the use of different materials for the shaft 102 and the handle 112. This construction allows the shaft 102 to be manufactured from a rigid material providing structural integrity while the handle 112 is manufactured from a softer or more resilient material providing grip comfort and security.

The surface finish of the integrated cricothyrotomy obturator 100 is configured to minimize friction during insertion and tube advancement while maintaining sufficient surface texture to prevent uncontrolled slippage. The distal tip assembly 108, tapered dilating section 110, and elongated flattened shaft 102 may have a relatively smooth surface finish to reduce tissue friction and facilitate cleaning. The proximal finger hold handle 112 and syringe holder 114 may have a matte or textured finish to enhance grip.

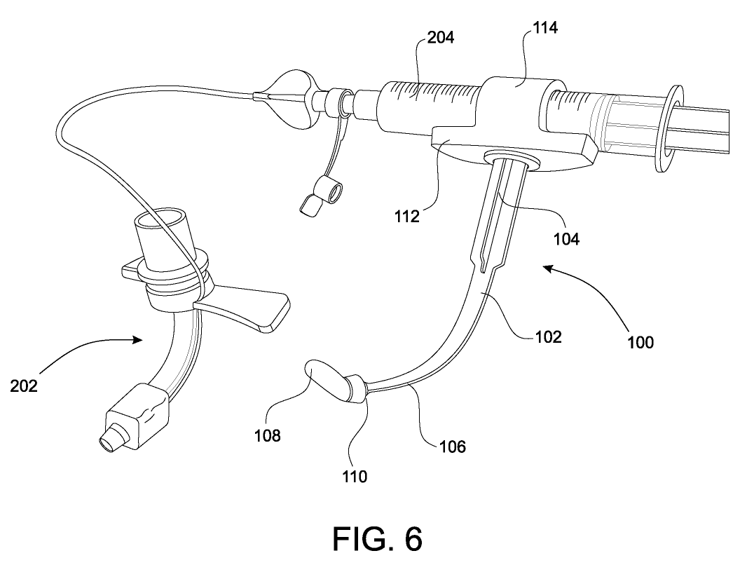

FIG. 6 depicts the cricothyrotomy obturator 100 with a syringe 204 disposed within the syringe holder 114. The syringe 204 is coupled to a cricothyrotomy tube 202. FIG. 7 depicts the cricothyrotomy obturator 100 disposed within the cricothyrotomy tube 202. The obturator 100 may be provided in a kit that includes the obturator 100 with a pre-loaded cricothyrotomy tube 202, a syringe 204 pre-positioned in the syringe holder 114, a scalpel for incision creation, and a neck strap for post-placement tube stabilization.

In operation, the integrated cricothyrotomy obturator 100 is used in conjunction with a standard cricothyrotomy tube 202 to establish emergency surgical airway access. The clinician first identifies the cricothyroid membrane by palpation and makes a horizontal stab incision through the membrane using a scalpel. The obturator 100, with the cricothyrotomy tube 202 pre-loaded over the elongated flattened shaft 102, is then inserted through the incision with the atraumatic distal tip 108 leading.

The upward angular orientation of the distal tip 108 causes the tip 108 to lift the superior edge of the incision as it passes through, eliminating the need for a separate tracheal hook. The clinician continues advancing the device until the tapered dilating section 110 passes through the incision, progressively enlarging the opening to accommodate the cricothyrotomy tube 202. The tube 202 is then advanced over the elongated flattened shaft 102 and through the dilated incision into the trachea.

The integration of multiple functions into a single device provides advantages in emergency situations where time is limited and equipment availability may be constrained. The elimination of separate tracheal hooks and dilating instruments reduces the number of items that must be gathered, opened, and manipulated during the procedure. The integrated syringe holder 114 ensures that cuff inflation equipment is immediately available without searching through equipment bags or supply carts.

The flattened-to-round transitional geometry of the device 100 represents a departure from conventional obturator designs that typically employ uniformly round cross-sections throughout their length. This transitional geometry enables the device 100 to perform the membrane-lifting function traditionally accomplished by a separate tracheal hook while simultaneously providing the dilating function traditionally accomplished by a separate bougie or dilator. The combination of these functions in a single instrument reduces procedural complexity and the potential for errors associated with instrument exchanges.

The curved profile of the elongated flattened shaft 102 matches the curvature of standard cricothyrotomy tubes, ensuring proper alignment between the obturator 100 and tube 202 throughout the insertion process. This matching curvature prevents binding or kinking that might occur if a straight obturator were used with a curved tube, and ensures smooth tube advancement without requiring the clinician to manipulate or rotate the device during insertion.

The obturator shaft 102 is dimensioned to receive and guide standard 6.0 mm cricothyrotomy tubes, establishing mechanical compatibility with existing airway management equipment. This interface allows the pre-loaded tube 202 to track along the obturator 100 and into the trachea in a single continuous motion. The integrated syringe holder 114 is configured to accept standard medical syringes, enabling cuff inflation while maintaining the clinician’s grip on the device assembly. This arrangement permits airway securement without requiring the clinician to release the device 100 or coordinate with additional personnel.

Various modifications and alterations to the integrated cricothyrotomy obturator 100 will be apparent to those skilled in the art. The dimensions, materials, and configurations described herein are illustrative and may be varied to accommodate different tube sizes, patient populations, or clinical requirements.

### Additional Considerations

As used herein any reference to "one embodiment" or "an embodiment" means that a particular element, feature, structure, or characteristic described in connection with the embodiment is included in at least one embodiment. The appearances of the phrase "in one embodiment" in various places in the specification are not necessarily all referring to the same embodiment.

Some embodiments may be described using the expression "coupled" and "connected" along with their derivatives. For example, some embodiments may be described using the term "coupled" to indicate that two or more elements are in direct physical or electrical contact. The term "coupled," however, may also mean that two or more elements are not in direct contact with each other, but yet still co-operate or interact with each other. The embodiments are not limited in this context.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, "or" refers to an inclusive or and not to an exclusive or. For example, a condition A or B is satisfied by any one of the following: A is true (or present) and B is false (or not present), A is false ( or not present) and B is true ( or present), and both A and B are true ( or present).

In addition, use of the "a" or "an" are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. An obturator for a cricothyrotomy procedure, the obturator comprising:
an elongated shaft comprising a proximal portion having a proximal end and a distal portion having a distal end, wherein the shaft is curved, and wherein the distal portion has a cross-sectional shape that is flat;
a distal tip assembly comprising a tapered dilating section directly coupled to the distal end of the shaft, and an atraumatic tip directly coupled to the tapered dilating section, wherein the atraumatic tip has a solid, flat cross-section and a rounded end, and wherein the tapered dilating section has a cross-sectional shape that gradually increases from a first size that is substantially equal to that of the flat cross-section to a second size having a round cross-section; and
a proximal handle assembly comprising a finger hold handle directly coupled to the proximal end of the shaft, and a syringe holder directly coupled to the finger hold handle, wherein the syringe holder has a cylindrical bore configured for retaining a syringe barrel therein.

2. The obturator of claim 1, wherein the distal tip assembly is formed as a single, unitary piece.

3. The obturator of claim 1, wherein the proximal portion of the shaft has a second cross-sectional shape that is different from the distal portion cross-sectional shape.

4. The obturator of claim 3, wherein the second cross-sectional shape is a cross.

5. The obturator of claim 1, wherein the distal tip assembly and the elongated shaft are formed as a single, unitary piece.

6. The obturator of claim 1, wherein the proximal handle assembly is formed as a single, unitary piece.

7. The obturator of claim 1, wherein the cross-sectional shape of the distal portion is a solid, flat rectangle.

8. An assembly for a cricothyrotomy procedure, wherein the assembly comprises:
a cricothyrotomy tube;
an obturator configured for being positioned within the cricothyrotomy tube; and
a syringe operatively coupled to the cricothyrotomy tube and configured for inflating a cuff on the cricothyrotomy tube,
wherein the obturator comprises:
an elongated shaft that is sized and shaped to fit entirely within the cricothyrotomy tube;
a distal tip assembly comprising a tapered dilating section directly coupled to a distal end of the shaft, and an atraumatic tip directly coupled to the tapered dilating section, wherein the atraumatic tip has a solid, flat cross-section and a rounded end, and wherein the tapered dilating section has a cross-sectional shape that gradually increases from a first size that is substantially equal to that of the flat cross-section to a second size that is round and has a diameter approximately equal to an inner diameter of the cricothyrotomy tube; and
a proximal handle assembly comprising a finger hold handle directly coupled to a proximal end of the shaft, and a syringe holder directly coupled to the finger hold handle, wherein the syringe holder has a cylindrical bore having an inner diameter that is approximately equal to an outer diameter of a barrel of the syringe.

9. The assembly of claim 8, wherein the cricothyrotomy tube is curved and the elongated shaft has a curved profile that matches that of the cricothyrotomy tube.

10. The assembly of claim 8, wherein the assembly has an assembled configuration wherein the elongated shaft of the obturator is positioned within the cricothyrotomy tube, the syringe is positioned in the syringe holder, the distal tip assembly of the obturator protrudes out of a distal end of the cricothyrotomy tube, and the proximal handle assembly is positioned proximally of a proximal end of the cricothyrotomy tube.

11. The assembly of claim 8, wherein the elongated shaft of the obturator has a proximal section comprising a first cross-sectional shape and a distal section comprising a second cross-sectional shape that is different from the first cross-sectional shape.

12. The assembly of claim 11, wherein the first cross-sectional shape is a cross and the second cross-sectional shape is solid and flat .

13. The assembly of claim 11, wherein the distal section of the elongated shaft has a solid, flat cross-sectional shape.

14. The assembly of claim 8, wherein the distal tip assembly is formed as a single, unitary piece.

15. The assembly of claim 8, wherein the distal tip assembly and the elongated shaft are formed as a single, unitary piece.

16. The assembly of claim 8, wherein the proximal handle assembly is formed as a single, unitary piece.
